(19) United States
(12) Patent Application Publication
(10) Pub. No.: US 2009/0182306 A1
(43) Pub. Date: Jul. 16, 2009

(54) Microneedle Devices and Methods of Drug Delivery or Fluid Withdrawal

(75) Inventors: Jeong Woo Lee, Atlanta, GA (US); Mark R. Prausnitz, Atlanta, GA (US)

Correspondence Address: SUTHERLAND ASBILL & BRENNAN LLP, 999 PEACHTREE STREET, N.E., ATLANTA, GA, 30309, US

(73) Assignee: Georgia Tech Research Corporation, Atlanta, GA (US)

(21) Appl. No.: 12/357,195

(22) Filed: Jan. 21, 2009

Related U.S. Application Data
(63) Continuation-in-part of application No. PCT/US07/74123, filed on Jul. 23, 2007.
(60) Provisional application No. 61/023,066, filed on Jan. 23, 2008.
(60) Provisional application No. 60/832,479, filed on Jul. 21, 2006.

(51) Int. Cl. A61M 5/00; B29C 39/42

(52) U.S. Cl. 604/506; 604/272; 264/101

(57) ABSTRACT

Microneedle devices and methods of manufacture and use thereof are provided. The devices may be used in controlled delivery of drug across or into a biological barrier, such as skin, or fluid withdrawal from a biological barrier. In one case, the device includes a base substrate which comprises a drug dispersed in a swellable matrix material; and one or more microneedles extending from the base substrate, wherein the one or more microneedles comprise a water-soluble or water-swellable material, wherein the one or more microneedles will dissolve or swell following insertion into the biological barrier, providing a transport pathway for the drug to pass from the base substrate into the biological barrier.

### CROSS-REFERENCE TO RELATED APPLICATIONS

This application is a continuation-in-part of International Application No. PCT/US07/074,123, filed Jul. 23, 2007, which claims benefit to U.S. Provisional Application No. 60/832,479, filed Jul. 21, 2006. This application also claims benefit to U.S. Provisional Application No. 61/023,066, filed Jan. 23, 2008. These applications are incorporated herein by reference.

### STATEMENT REGARDING FEDERALLY SPONSORED RESEARCH OR DEVELOPMENT

This invention was made with U.S. government support under Contract No. 8R01EB00260 awarded by the National Institutes of Health. The U.S. government has certain rights in the invention.

### BACKGROUND OF THE INVENTION

This invention is generally in the field of devices and methods for the controlled transport of molecules across skin or other tissue barriers, such as for drug delivery or sampling of biological fluids.

Numerous drugs and therapeutic agents have been developed in the battle against disease and illness. A frequent limitation to the effective and efficient use of these drugs, however, is their delivery, that is, how to transport the drugs across biological barriers in the body (e.g., the skin, the oral mucosa, the blood-brain barrier), which normally do not transport drugs at rates that are therapeutically useful or optimal.

Transdermal drug delivery systems have been shown to be an effective alternative drug pathway for local or systemic drug delivery. Although these systems provide numerous advantages to oral drug delivery routes, development of transdermal delivery devices has been limited by the diffusion of drugs across the stratum corneum of the skin.

To address these problems, microneedles have been developed employing a variety of different fabrication processes and application strategies and may be classified according to the drug delivery strategy. One concept uses microneedles to break the stratum corneum to create pathways through which a drug may enter and thereafter applying a patch to the skin as a drug reservoir. Another concept uses hollow microneedles as micro ducts for the flow of drug in liquid formulations. Still another approach uses coated microneedles to deliver small amounts of drug loaded onto the microneedle surface. While each of these approaches provides improved drug delivery across the stratum corneum, there still remains a need for improved transdermal drug delivery devices. The first two approaches may be limiting in their requirement of an additional feature or step for drug delivery, while the third approach may be limiting in the amount of drug that may be loaded onto the surface of the coated microneedles. Accordingly, there remains a need to provide improved microneedle devices and methods, particularly for simple and effective transdermal delivery of wide ranges and/or relatively large volumes of drug.

In addition, it would be desirable to have microneedle array devices providing bolus and/or sustained delivery of a macromolecular drug with a relatively large range of therapeutic dose. It would also be desirable to provide a microneedle device with the drug in a stable encapsulated form.

Microneedles also have been proposed for minimally-invasive withdrawal of biological fluids from patients for diagnostic purposes. Some of these devices include multiple parts, which may be fragile, costly to produce, and/or difficult to use properly. It would be desirable to provide improved devices which can be made relatively inexpensively and which are relatively simple to use and effective.

### SUMMARY OF THE INVENTION

Microneedle devices and methods of use thereof are provided, along with methods of manufacturing the microneedle devices. The devices and methods address one or more of the drawbacks associated with prior microneedle devices.

In one aspect, a device is provided for sustained delivery of drug across or into a biological barrier. In one embodiment, the device includes a base substrate which comprises a drug dispersed in a swellable matrix material; and one or more microneedles extending from the base substrate, wherein the one or more microneedles comprise a water-soluble or water-swellable material, wherein the one or more microneedles will dissolve or swell following insertion into the biological barrier, providing a transport pathway for the drug to pass from the base substrate into the biological barrier, and wherein the base substrate is adapted to swell following insertion of the one or more microneedles into the biological barrier. The one or more microneedles may further include a drug dispersed in the water-soluble or water-swellable material.

In one embodiment, the water-soluble or water-swellable material of the microneedles comprises a polysaccharide or a derivative thereof. The water-soluble or water-swellable material may comprise a cellulose derivative. The water-soluble or water-swellable material may become a hydrogel upon insertion into the biological barrier. In certain embodiments, the water-soluble or water-swellable material may include carboxymethyl cellulose, hydroxypropylmethyl cellulose, amylopectin, starch derivatives, hyaluronic acid, or a combination thereof.

The matrix material of the base substrate may be polymeric, such as a biocompatible or biodegradable polymer. The polymeric matrix material may comprise a water-soluble or water-swellable material, which may be the same as or different from the water-soluble or swellable material of the one or more microneedles.

The one or more microneedles may each be solid or hollow. In one embodiment, the microneedles each have a length between about 10 μm and about 1500 μm. In one embodiment, the microneedles each have a maximum width between about 10 μm and about 500 μm. The microneedles may have a pyramidal shape.

In one embodiment, the microneedle device includes a backing layer attached to the base substrate distal to the one or more microneedles. In one case, the backing layer has an annular region which surrounds the one or more microneedles. This annular region may include an adhesive substance for contacting a patient's skin or other tissue.

In a particular embodiment, a microneedle array for drug delivery is provided that includes a base substrate comprising a first drug dispersed in a swellable polymeric matrix material; a plurality of microneedles extending from the base substrate, wherein the plurality of microneedles comprises a water-soluble or water-swellable material in which a second drug may be dispersed, wherein the plurality of microneedles will dissolve following insertion into a biological barrier, providing a transport pathway for the first and second drugs to pass into the biological barrier, and wherein the base substrate is adapted to swell following insertion of the one or more microneedles into the biological barrier. The first drug and the second drug may be the same drug or different drugs. In certain variations of this embodiment, the water-soluble or water-swellable material of the plurality of microneedles may comprise carboxymethyl cellulose, hydroxypropylmethyl cellulose, amylopectin, starch derivatives, hyaluronic acid, or a combination thereof. In certain variations of this embodiment, the polymeric matrix material of the base substrate may comprise carboxymethyl cellulose, hydroxypropylmethyl cellulose, amylopectin, starch derivatives, or a combination thereof. In one embodiment of these microneedle devices, the drug is a peptide or protein. In one embodiment, the drug is a vaccine. In one embodiment, the drug is a small molecule with a molecular mass less than 2000 Da or, in some cases, less than 1000 Da or 500 Da. In an embodiment, an adhesive substance coating is provided on at least a portion of the surface of the base substrate between/among the microneedles.

In another aspect, a method is provided for delivering a drug across or into the skin or another biological barrier. In one embodiment, the method includes the steps of (i) inserting the one or more microneedles of the device into the biological barrier, to create one or more holes in the biological barrier; (ii) dissolving or swelling the one or more microneedles in the biological barrier; and (iii) transporting the drug from the swellable base substrate through the holes and into the biological barrier. In one particular embodiment, the method further includes dissolving or swelling the one or more microneedles to release the drug from the one or more microneedles into the biological barrier. In a certain embodiment, the drug from the microneedles is substantially released within a period from about a few seconds to about one hour after insertion of the one or more microneedles into the biological barrier. In another particular embodiment, the drug from the base substrate is substantially released within a period from about one hour to about three days after insertion of the one or more microneedles into the biological barrier.

In one embodiment, the one or more microneedles of the device further include a drug (i) dispersed in the water-soluble or water-swellable material, (ii) coated onto the one or more microneedles, or (iii) dispersed in the water-soluble or water-swellable material and coated onto the one or more microneedles. In the latter case, the drug dispersed in the water-soluble or water-swellable material may be the same as or different from the drug coated onto the microneedle.

In still another embodiment, the method includes the steps of: (a) providing a microneedle device that includes (i) a base substrate which comprises a drug dispersed in a swellable polymeric matrix material, and (ii) a plurality of microneedles extending from the base substrate; (b) inserting the microneedles into the biological barrier, to create a plurality of holes in the biological barrier; (c) permitting aqueous fluids from the biological barrier to flow through the holes to hydrate and swell the base substrate, thereby creating fluid pathways within the base substrate for diffusion of the drug within the base substrate; and (d) allowing the drug to diffuse from the base substrate through the holes and into the biological barrier. In a certain embodiment, the one or more microneedles may remain partially intact during the hydrating and swelling of the base substrate.

In yet another aspect, a method is provided for extracting a fluid from or through a biological barrier. In one embodiment, the method includes: (a) providing a microneedle device that includes (i) a base substrate which comprises a water-swellable polymeric material, and (ii) one more microneedles extending from the base substrate, which one or more microneedles comprise a water-soluble or water-swellable material; (b) inserting the one or more microneedles into the biological barrier, to create a corresponding one or more holes in the biological barrier; and (c) withdrawing fluid from the biological barrier through the one or more holes and into the base substrate. For example, the biological barrier may comprise the skin or sclera of a human, and the fluid may comprise interstitial fluid or vitreous humor and solutes therein. In certain embodiments, the method further comprises analyzing the composition of the fluid, or a part thereof.

In still another aspect, a method is provided for making a microneedle device. In one embodiment, the method includes (a) providing an inverse mold for at least one microneedle, the mold having base surface in which are located one or more concavities, each in the shape of a microneedle; (b) providing a microneedle structural material in a fluidized form, which comprises a water-soluble or -swellable material; (c) using centrifugation or vacuum (or other pressure source) to force the fluidized structural material into the one or more concavities; (d) hardening the structural material into the form of one or more microneedles; (e) forming a base substrate connected to the one or more microneedles, wherein the base substrate comprises a drug dispersed in a polymeric matrix material, which may be a swellable polymeric matrix material; and (f) releasing the one or more microneedles from the inverse mold. In one embodiment, the base substrate and the one or more microneedles are formed together in one step by hardening of the fluidized structural material. In one embodiment, the fluidized structural material further comprises a solvent and the hardening step further comprises evaporating the solvent. In a certain embodiment, the inverse mold comprises a plurality of the concavities. In one embodiment, the one or more microneedles do not comprise a drug.

### BRIEF DESCRIPTION OF THE DRAWINGS

FIG. 1 is a cross-sectional, side view of a microneedle device according to one embodiment.

FIG. 2 is a cross-sectional, side view of a microneedle device according to another embodiment.

FIG. 3 is a cross-sectional side view of a microneedle patch device according to one embodiment.

FIG. 4 illustrates a method for using an embodiment of the microneedle device according to one embodiment.

FIG. 5 illustrates a method for using an embodiment of the microneedle device according to another embodiment.

FIG. 6 illustrates a process for the fabrication of a microneedle device according to one embodiment.

FIG. 7 illustrates a process for the fabrication of a microneedle device according to another embodiment.

FIGS. 8A-B are graphs of in vitro release profiles with Franz cell.

FIG. 9 is a graph of transdermal flux, as cumulative amount of sulforhodamine released over time, with a microneedle patches inserted into human cadaver skin, the patch having either a carboxymethylcellulose matrix or amylopectin matrix.

FIGS. 10-11 are cross-sectional views a microneedle device that includes a separate reservoir for containing (FIG. 10) and releasing a fluid that is intended to wet and swell the base substrate (FIG. 11), according to one embodiment.

FIG. 12 is a graph showing concentration of human growth hormone present in serum over time with microneedle patches inserted into the skin of hairless rats shown in comparison to the subcutaneous injection of human growth hormone into hairless rats, the patch having either carboxymethylcellulose microneedles or microneedles comprising both carboxymethylcellulose and a disaccharide.

### DETAILED DESCRIPTION OF THE PREFERRED EMBODIMENTS

Microneedle devices for the delivery of drugs across or into a biological tissue/barrier are provided, which advantageously may overcome limitations and deficiencies associated with prior art devices. The devices may provide sustained release from a drug storage volume that advantageously is not limited to the volume of the microneedles alone, in a simple construction which is easy to use. In one embodiment, the microneedle device is in the form of a transdermal patch. In one aspect, the single-use microneedles beneficially leave behind no sharp and rigid needles for disposal or concern about unauthorized re-use. Methods for the manufacture and use of microneedle devices are also provided.

As used herein, the terms “comprise,” “comprising,” “include,” and “including” are intended to be open, non-limiting terms, unless the contrary is expressly indicated.

### Microneedle Devices

In one aspect, a microneedle device is provided for sustained release of drug across or into a biological barrier. The biological barrier may be a biological tissue of a patient in need of the drug. The patient may be a human or other mammal, for example. The microneedle device may facilitate transport of one or more drugs through a barrier layer, such as the stratum corneum, and into underlying dermal tissues. The term “biological barrier” may include essentially any cells, tissues, or organs, including the skin or parts thereof, mucosal tissues, vascular tissues, lymphatic vessels, ocular tissues (e.g., cornea, conjunctiva, sclera), and cell membranes. The biological tissue may be in humans or other types of animals (particularly mammals), as well as in plants, insects, or other organisms, including bacteria, yeast, fungi, and embryos. Human skin and ocular tissues may be of particular use with the present devices and methods.

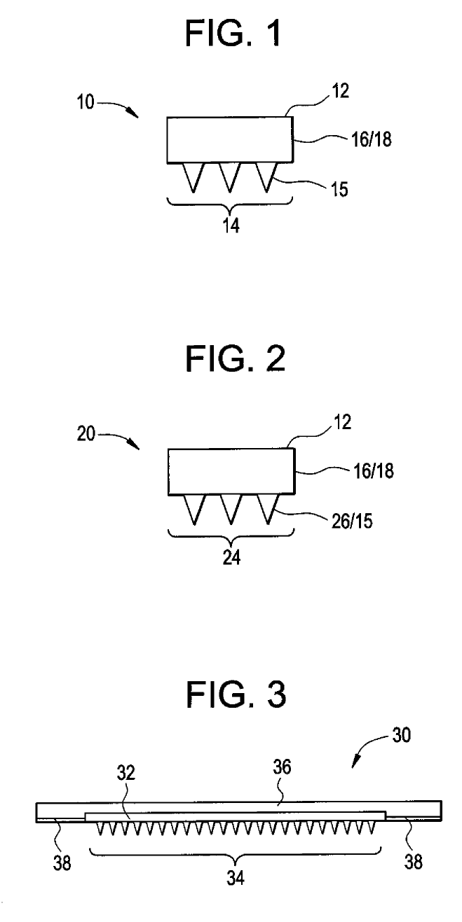

In one embodiment, the device includes a swellable base substrate which comprises a drug dispersed in a matrix material; and one or more microneedles extending from the swellable base substrate, wherein the one or more microneedles include, or consist essentially of, a water-soluble or water-swellable material, and wherein the one or more microneedles will dissolve or swell following insertion into the biological barrier, providing a transport pathway for the drug to pass from the base substrate into the biological barrier. The matrix material may be a polymer. The drug transport may be by diffusion, alone or enhanced by an active mechanism known in the art, such as electric fields or ultrasound. FIG. 1 shows one embodiment of a microneedle device 10 which includes a swellable base substrate 12 and three microneedles 14 extending from the base substrate. The base substrate 12 includes drug 16 dispersed in a polymeric matrix material 18. The microneedles 14 include a water-soluble or -swellable material 15. In various embodiments, the one or more microneedles may be solid or hollow, may have a length between about 10 μm and about 1500 μm, and may have a maximum width between about 10 μm and about 500 μm. In a preferred embodiment, the one or more microneedles taper to a sharp tip, which may have a pyramidal shape. In a preferred embodiment, the microneedle has an aspect ratio between about 1.5 and 2.5, more particularly between about 1.8 and 2.2, or about 2.0. This range of aspect ratio may be particularly useful for CMC, certain polysaccharides, or other mechanically weak biomaterials.

In another embodiment, the one or more microneedles further include a drug, which may be dispersed in all of, or a portion of, the water-soluble or water-swellable material. The drug provided in the base substrate may be the same as or different from the drug provided in the one or more microneedles. FIG. 2 shows one embodiment of a microneedle device 20 which includes a swellable base substrate 12 and three microneedles 24 extending from the base substrate. The base substrate 12 includes drug 16 dispersed in a matrix material 18. The matrix material may be polymeric. The microneedles 24 include drug molecules 26 dispersed in the water-soluble or -swellable material 15. In one embodiment, the one or more microneedles may provide a dose of a drug for immediate release (e.g., by dissolving rapidly upon insertion into the biological tissue) while the base substrate provides a sustaining or maintenance dose of the same drug (e.g., due to the greater time needed for the drug to diffuse from base substrate through the holes in the biological tissue). Alternatively the second drug could be a different drug for the same or a different indication as that of the first drug.

In one embodiment, a microneedle array device is provided for drug delivery. The array device may be part of a transdermal patch. The array device may include a base substrate comprising a first drug dispersed in a swellable matrix material; a plurality of microneedles extending from the base substrate, wherein the plurality of microneedles comprise a water-soluble or water-swellable, or otherwise dissolvable material in which a second drug is dispersed, wherein the plurality of microneedles will dissolve and/or swell following insertion into a biological barrier, providing a transport pathway for the first and second drugs to pass into the biological barrier. The matrix material may be polymeric. The first drug and the second drug may be the same drug, or they may be different from one another.

In various embodiments, the device may include features for inserting the one or more microneedles into a biological tissue. This feature may be include mechanical or electrical parts, or alternatively, may include a rigid or pliable structure for manually pressing the microneedle into, and the base substrate structure against, skin or other tissues. For example, the device may include a backing layer attached to the base substrate distal to the one or more microneedles. In one case, the backing layer may have an annular region which surrounds the one or more microneedles, wherein the annular region includes an adhesive substance for contacting a patient's skin. Alternatively, or in addition, an adhesive substance is provided (e.g., in a thin film) on the surface of the base substrate, e.g., between some or all of the microneedles. In a preferred embodiment, the backing layer is substantially impervious to the drug in the base substrate, to water vapor, and/or to physiological fluids from the biological barrier. The backing layer may stretch or deform to accommodate swelling/expansion of base substrate during use. For example, it may include an elastomeric film. FIG. 3 illustrates one embodiment of a microneedle patch device 30 which includes a swellable base substrate 32 from which an array of microneedles 34 extend. The base substrate includes a drug for release. The device 30 further includes backing layer 36 with adhesive 38 for securing the patch to a skin surface during drug delivery. Suitable adhesive substances, such as pressure sensitive adhesives, are well known in the art of adhesive bandages and transdermal drug delivery patches.

Microneedles and Base Substrate

The one or more microneedles extend from the base substrate. The microneedle is formed/constructed of biocompatible materials that will degrade and/or dissolve, or swell, in the biological barrier, e.g., in physiological fluids present in the biological barrier at the site of insertion of the microneedle. The material(s) of construction and the dimensions of the microneedle are selected to provide, among other things, the mechanical strength to remain substantially intact while being inserted into the skin or into other biological barrier.

In one embodiment, the material of construction of the microneedle includes a water soluble material. As used herein, a “water soluble” material is one that dissolves, hydrolyzes, or otherwise breaks down or disintegrates in water or in contact with an aqueous physiological fluid, such as blood, tears, interstitial fluid, mucus, etc., over a period of time following insertion into a biological barrier. The period of time may be rapid, e.g., less than 10 seconds, less than 1 minute, less than 5 minutes, less than 10 minutes, less than 30 minutes, less than 1 hour, less than 4 hours, less than 8 hours, less than 12 hours, or less than 24 hours. In a certain embodiment, the water soluble material comprises a polymer. In one case, it is a polysaccharide or derivative thereof.

In another embodiment, the material of construction of the microneedle includes a water-swellable material. As used herein, “water-swellable” refers to materials which imbibe aqueous fluids that are in contact therewith, causing the materials to expand. In one embodiment, the material comprises a hydrogel. Hydrogels may be uncrosslinked or crosslinked. Uncrosslinked hydrogels are able to absorb water but may not dissolve due to the presence of hydrophobic and hydrophilic regions. Covalently crosslinked hydrogels may include networks of hydrophilic polymers, including water-soluble polymers. The material may be initially dry and then become a hydrogel upon insertion into the biological barrier. In a certain embodiment, the material is a cross-linked polymer. In various embodiments, the water-swellable material may comprise a polyacrylic acid known in the art.

In one embodiment, the microneedle includes a combination of a water-swellable material and a water-soluble material. The combination may be, for example, a mixture of the materials or a layered structure comprising at least one layer of the water-soluble material being provided on top of at least one layer of the water-swellable material.

The base substrate may be made of the same water-swellable materials described herein for forming the microneedles, or it may comprise one of the water-soluble materials listed that would swell without extensive dissolution under the particular conditions used. Alternatively, the base substrate may be made of a different swellable material.

The water-soluble and/or water-swellable materials may comprise a polysaccharide or a derivative thereof. In one embodiment, the material is a biocompatible cellulose derivative. In certain embodiments, the water soluble material may be selected from carboxymethyl cellulose, hydroxypropylmethyl cellulose, amylopectin, starch derivatives, hyaluronic acid, or a combination thereof.

The water-soluble and/or water-swellable materials also may comprise a polysaccharide, such as alginate, amylose, amylopectin, carrageenan, carboxymethyl cellulose, dextran, gellan, guar gum, polysaccharide conjugate vaccines, hydroxyethyl cellulose, hydroxypropyl cellulose, hyaluronic acid, starch derivatives, xantan, xyloglucan, chitosan-based hydrogel, peptidoglycan, and progeoglycans.

The water-soluble and/or water-swellable materials also may comprise a carbohydrate, such as glucose, maltose, lactose, fructose, sucrose, galactose, glucosamine, galactosamine, muramic acid, glucruronate, gluconate, fucose, and trehalose.

The water-soluble and/or water-swellable materials also may comprise a synthetic polymer, such as polyvinyl alcohol, polyvinlypyrrolidine, polyethyleneglycol, and polyoxyethylene derivatives. In other cases, the water-soluble or -swellable material may comprise a polypeptide, such as polyvinyl amine or poly(L-lysine).

The water-soluble and/or water-swellable materials may include or consist of a water-soluble or biodegradable polymer. Examples of suitable biodegradable polymers may include poly(lactide)s, poly(glycolide)s, poly(lactide-co-glycolide)s, polyanhydrides, polyorthoesters, polyetheresters, polycarpolactones, polyesteramides, poly(butyric acid)s, poly(valeric acid)s, polyhydroxyalkanoates, degradable polyurethanes, copolymers thereof, and blends thereof. Alternatively, the water-soluble and/or water swellable material may be a non-degradable polymer. Examples of non-degradable polymers include polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends, and copolymers thereof. In certain embodiments, hydrogel materials such as carboxymethyl cellulose (CMC), hydroxypropylmethyl cellulose (HPMC), amylopectin, starch derivatives, hyaluronic acid, or a combination thereof may be used as the water-soluble and/or water-swellable material.

In a preferred embodiment, the microneedles and the base substrate comprise carboxymethyl cellulose.

The base substrate includes one or more drugs. The drug may be located throughout the base substrate material or provided in a sub-component thereof. The drug may be dispersed in the polymer. As used herein, the phrase “dispersed in the polymer” refers to various forms of the drug, including where the drug is dissolved, where the drug is a separate solid or liquid phase, or where the drug is encapsulated into a further material that is within the polymer matrix. For instance, microparticles or nanoparticles of drug may be microencapsulated or nanoencapsulated within another release controlling substance (e.g., a biocompatible polymer, such as a hydrophobic or amphiphilic polymer) and these microparticles or nanoparticles may be dispersed within the polymer matrix material of the base substrate.

Advantageously, the base substrate simultaneously serves as a platform for the microneedles and storage reservoir for the drug. Beneficially the drug may be stored in a substantially dry, solid form, encapsulated by the matrix material. The drug and polymeric matrix may be a solid solution. In one embodiment, the drug may comprise between about 0.1% and about 70%, such as between 1% and 50% (e.g., between 1 and 25% or between 1 and 10%), by weight of the base substrate and microneedles. Higher or lower loadings may be used, depending upon the particular drug and particular polymeric matrix material used.

In one embodiment, the swellable base substrate comprises a material that swells when exposed to fluid and will not substantially dissolve in that fluid under the intended operating conditions. In certain embodiments, the base substrate comprises a crosslinked polymer being sufficiently crosslinked to prevent dissolving, but weakly enough crosslinked to permit swelling. In certain embodiments, the base substrate comprises a material that has a very low solubility in the fluid, such that only a small portion of the base substrate material dissolves. In certain embodiments, the microneedles are designed such that only a small amount of fluid enters the base substrate which limits solubilization of the base substrate material.

The base substrate may include a combination of a water-swellable material and a water-soluble material. The combination may be, for example, a mixture of the materials or a layered structure comprising at least one layer of the water-soluble material being provided adjacent at least one layer of the water-swellable material. The base substrate may be made of the same material as that forming the microneedle or it may be made of a different material.

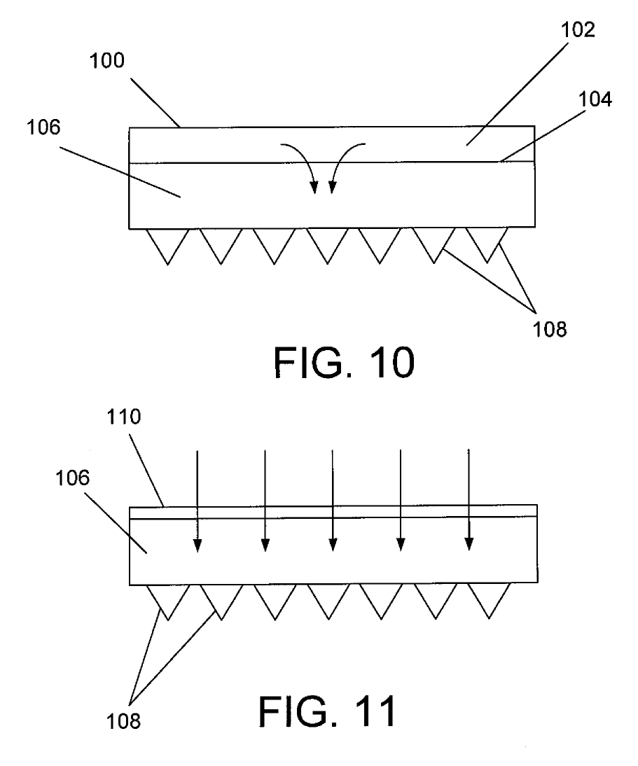

A variety of different fluids may be used to swell the base substrate. In some embodiments, the fluid comprises fluid from the tissue into which the microneedles were inserted, including interstitial fluid or sweat. The fluid may be aqueous. In other embodiments, the fluid may be provided from a source other than tissue. The fluid that dissolves the microneedles may be the same fluid that swells the base substrate, or it may be a different fluid. In some embodiments, fluid may be stored in a separate reservoir of the microneedle device as illustrated in FIG. 10. In this embodiment, a reservoir 102 of the fluid is contained between the backing 100 and a membrane 104. The membrane 104 may separate the fluid in the reservoir 102 from the swellable matrix material of base substrate 106. In certain embodiments, pressure applied to the backing 100, such as when applying pressure to press the microneedles 108 into a biological barrier, causes the membrane 104 to rupture or otherwise forces the fluid contained in the reservoir 102 into the matrix material 106, as indicated by the arrows in FIG. 10. This causes the base substrate 106 to swell, and the drug contained in base substrate 106 may thereafter be transmitted through channels created by the microneedles 108.

In other embodiments, a user may apply the fluid to the backing as illustrated in FIG. 11. In this embodiment, the backing 110 is permeable to the fluid such that the fluid may pass through the backing 110 and into the base substrate 106. In certain embodiments, the fluid may be water or an aqueous solution. As such, the user may first apply the device such that the microneedles 108 penetrate a biological barrier. The user may then apply water or another fluid to the outer surface of the backing 110 to cause the matrix material 106 to swell. The drug contained in the matrix material 106 may thereafter be transmitted through channels created by the microneedles 108. In various embodiments, the microneedles are used to limit the amount of fluid that enters the base substrate or otherwise prevent substantial dissolution of the base substrate. One approach is to limit the size and number of transport pathways made in the tissue for fluid transport into the base substrate so as to avoid sufficient fluid entering the base substrate to cause significant dissolution. Another approach is to provide a sufficiently large amount of base substrate material to prevent substantial dissolution. Another approach is to design the device to permit evaporation, or removal by another mechanism, of the fluid from the base substrate. In this way, although large amounts of fluid may enter the base substrate, it leaves the base substrate at a sufficiently fast rate that not enough fluid collects in the base substrate to cause substantial dissolution of the base substrate material. Another approach is to add buffering agents to maintain the pH at conditions under which the base substrate material is less soluble in the fluid and thereby prevent substantial dissolution. Another approach is to have a base substrate made of multiple materials.

In some embodiments, the microneedles and their base substrate may be made of different materials. These different materials may have different chemical composition, e.g., can be polymers made of different monomeric units. Alternatively, the materials may be different materials that have similar chemical compositions, e.g., can be polymers made of the same monomeric units, but (i) the monomers are present in different ratios in the microneedles versus the base substrate, (ii) the molecular weight of the polymers in the microneedles is different from the base substrate, (iii) the degree of crosslinking is different in the microneedles versus the base substrate. Alternatively, the materials may have the same chemical composition, but (i) have different structure or properties (e.g., crystalline versus amorphous), (ii) be present in combination with other materials that are different or are at different compositions in the microneedles versus the base substrate, (iii) be designed to be exposed to fluid under different conditions, such that the fluid contacting the microneedles does so under conditions that substantially dissolve the microneedles and such that the fluid, which may be the same or different fluid, contacting the base substrate does so under conditions that do not substantially dissolve the base substrate and do substantially swell the base substrate.

Microneedle devices having dissolvable microneedles and a swellable base substrate may be fabricated using various methods in order to give the microneedles and the base substrate different properties to perform their designated functions. One approach involves a two-step fabrication process. As a first step, the microneedles are fabricated under conditions that produce dissolvable microneedles. As a second step, the base substrate is fabricated under conditions that produce a swellable base substrate. The second step may be performed such that the base substrate is attached to the microneedles as an integral part of the process. For example, the cavities of a mold may be filled with material to form the dissolvable microneedles. Subsequently, the top surface of the mold may be covered with material to form the swellable base substrate. In this way, the microneedles and the base substrate form an integrated device that can be removed intact from the mold. Alternatively, the first step of forming the dissolvable microneedles and the second step of forming the swellable base substrate may be performed separately, such as by using separate molds. A third step may then be performed to connect the microneedles to the base substrate.

Microneedle devices with dissolvable microneedles and a swellable base substrate may be fabricated as a single-step process. In this case, the different properties of the dissolvable microneedles and the swellable base substrate can be created by a self-assembly process. For example, a mixture of materials may be cast onto a microneedle mold, where the material(s) needed for the microneedles are more dense than the material(s) needed for the base substrate. There may be a phase separation between these two set of materials, but this is not necessary. By gravity, pressure, centrifugation or other forces, the material(s) needed for the microneedles preferentially travel to the microneedle cavities in the mold and the material(s) needed for the base substrate preferentially travel to the external surface of the mold to form the base substrate. In addition to gravity, this separation may be performed using materials with different electric, magnetic or other properties and exposing them to suitably oriented electric, magnetic or other fields, respectively. The surface properties of the mold within the cavities and on the external surface also may be controlled to preferentially permit or exclude materials from entering the cavities and depositing on the external surface due to charge, hydrophobicity or other physicochemical forces. A single-step process also may be used where there is no separation or gradient between the cavities and external surface of the mold. In that scenario, the materials in the microneedles and the base substrate may be the same, but the design/geometry of the microneedle device gives the dissolvable microneedles and the swellable base substrate their correct properties.

In an alternative embodiment, the microneedles may be formed as a composite of two or more degradable or dissolvable materials. The materials may be combined heterogeneously or as a homogeneous mixture. For example, in the heterogeneous embodiments, the materials may be built up in layers, such that the composition varies along the shaft of the microneedle, or the microneedle may have a core of a first material with a coating of a second material formed onto the core. Additional layers of the first or second material may then be included.

The microneedle may have a straight or tapered shaft. In one embodiment, the diameter of the microneedle is greatest at the base end of the microneedle and tapers to a point at the end distal the base. The microneedle can also be fabricated to have a shaft that includes both a straight (i.e., untapered) portion and a tapered portion. The microneedles can be formed with shafts that have a circular cross-section in the perpendicular, or the cross-section can be non-circular. In a preferred embodiment, the microneedle has a pyramidal shape, with a square or triangular base. The tip portion of the microneedles can have a variety of configurations. The tip of the microneedle can be symmetrical or asymmetrical about the longitudinal axis of the shaft. The tips may be beveled, tapered, squared-off, or rounded. The tip portion generally has a length that is less than 50% of the total length of the microneedle.

The dimensions of the microneedle, or array thereof, are designed for the particular way in which it is to be used. The length of the microneedle typically is selected taking into account both the portion that would be inserted into the biological barrier and the base portion that would remain uninserted. In various embodiments, the microneedle may have a length of about 10 μm to about 1500 μm. In an embodiment, the microneedle may have a length of about 50 μm to about 1500 μm, about 150 μm to about 1500 μm, about 300 μm to about 1500 μm, about 300 μm to about 1000 μm, or about 300 to about 750 μm. In one embodiment, the length of the microneedle is about 500 μm. In various embodiments, the base portion of the microneedle has a maximum width or cross-sectional dimension of about 10 μm to about 500 μm, about 50 μm to about 400 μm, or about 100 μm to about 250 μm. For a hollow microneedle, the maximum outer diameter or width may be about 50 μm to about 400 μm, with an aperture diameter of about 5 μm to about 100 μm. The microneedle may be fabricated to have an aspect ratio (width:length) of about 1:1 to about 1:10. Other lengths, widths, and aspect ratios are envisioned.

In various embodiments, the microneedle device includes an array of two or more microneedles. For example, the device may include an array of between 2 and 1000 (e.g., between 4 and 250 or between 10 and 100) microneedles. An array of microneedles may include a mixture of different microneedles. For instance, an array may include microneedles having various lengths, base portion diameters, tip portion shapes, spacings between microneedles, drug coatings, material composition, etc.

The single microneedle or array of two or more microneedles may extend from the base substrate of the microneedle device at any angle suitable for insertion into the biological barrier. In a particular embodiment, the base substrate of the microneedle device may be a substantially planar foundation from which the one or more microneedles extend, typically in a direction normal (i.e., perpendicular or ‘out-of-plane’) to the foundation. Alternatively, the microneedles may be fabricated on the edge of the base substrate ‘in-plane’ with the substrate. In one case, the microneedles may be fabricated with a flexible base substrate capable of conforming to the shape of the surface of the biological barrier.

Drugs

A wide range of drugs may be formulated for delivery with the present microneedle devices and methods. As used herein, the term “drug” is used broadly to refer to any prophylactic, therapeutic, or diagnostic agent, or other substance that may be suitable for introduction to biological tissues, including pharmaceutical excipients and substances for tattooing, cosmetics, and the like. In one embodiment, the drug is a substance having biological activity, e.g., a therapeutic or prophylactic agent. The drug may be a prodrug. The drug may be formulated with one or more excipient materials, such as a pharmaceutically acceptable excipient. The drug may be provided in various forms including solids, liquids, liquid solutions, gels, hydrogels, solid particles (e.g., microparticles, nanoparticles), or combinations thereof. The drug may comprise small molecules, large (i.e., macro-) molecules, or a combination thereof.

Non-limiting examples of suitable drugs include amino acids, vaccines, antiviral agents, DNA/RNA, gene delivery vectors, interleukin inhibitors, immunomodulators, neurotropic factors, neuroprotective agents, antineoplastic agents, chemotherapeutic agents, polysaccharides, anti-coagulants, antibiotics, analgesic agents, anesthetics, antihistamines, anti-inflammatory agents, and vitamins. The drug may be selected from suitable proteins, peptides and fragments thereof, which can be naturally occurring, synthesized or recombinantly produced.

A variety of other pharmaceutical agents known in the art may be formulated for administration via the microneedle devices described herein. Examples include β-adenoreceptor antagonists, miotics, sympathomimetics, carbonic anhydrase inhibitors, prostaglandins, anti-microbial compounds, including anti-bacterials and anti-fungals, anti-viral compounds, aldose reductase inhibitors, anti-inflammatory and/or anti-allergy compounds, local anesthetics, cyclosporine, diclofenac, urogastrone and growth factors such as epidermal growth factor, mydriatics and cycloplegics, mitomycin C, and collagenase inhibitors.

In a particular embodiment, the drug may be a vaccine and the water-soluble material may include a material which degrades into adjuvants useful for the vaccine. Examples of such materials known in the art include polyphosphazenes and CpG oligonucleotides.

### Methods of Using the Microneedle Devices

In another aspect, a method is provided for delivering a drug across or into a biological barrier. In one embodiment, the method includes (i) providing a device that includes a swellable base substrate which comprises a drug dispersed in a polymeric matrix material, and one or more microneedles extending from the base substrate, wherein the one or more microneedles comprises a water-soluble, or water-swellable material; (ii) inserting the one or more microneedles into a biological barrier, to create one or more holes (i.e., transport pathways) in the biological barrier; (iii) dissolving and/or swelling the one or more microneedles in the biological barrier; and (iv) allowing the drug to pass (e.g., diffuse or otherwise be driven) from the base substrate through the holes and into the biological barrier.

In another embodiment, the method includes (i) providing a device that includes a swellable base substrate which comprises a drug dispersed in a polymeric matrix material, and one or more microneedles extending from the base substrate, wherein the one or more microneedles comprises a water-soluble, or water-swellable material and a drug dispersed therein; (ii) inserting the one or more microneedles into the biological barrier, to create one or more holes in the biological barrier; (iii) dissolving and/or swelling the one or more microneedles in the biological barrier to release the drug from the one or more microneedles; and (iv) allowing the drug to diffuse (or be driven) from the base substrate through the holes and into the biological barrier.

In one case, the drug from the one or more microneedles may be substantially released within a period from about a few seconds to about one hour after insertion of the one or more microneedles into the biological barrier. In the same or another case, the drug from the base substrate is substantially released within a period from about one hour to about three days after insertion of the one or more microneedles into the biological barrier. The devices described herein can provide both rapid and sustained release of drug.

In a certain embodiment, the method for delivering a drug includes providing a microneedle array device that includes (i) a swellable base substrate which comprises a drug dispersed in a polymeric matrix material, and (ii) a plurality of microneedles extending from the base substrate; inserting the microneedles into the biological barrier, to create a plurality of holes in the biological barrier; permitting aqueous fluids from the biological barrier to flow through the holes to hydrate and swell the base substrate, thereby creating fluid pathways within the base substrate for diffusion of the drug within the base substrate; and transporting the drug from the base substrate through the holes and into the biological barrier. The one or more microneedles may remain partly intact during the hydration and swelling of the base substrate. The drug transport may occur solely or partially by diffusion. Transport may be enhanced, e.g., by the use of electrical fields or ultrasound techniques known in the art.

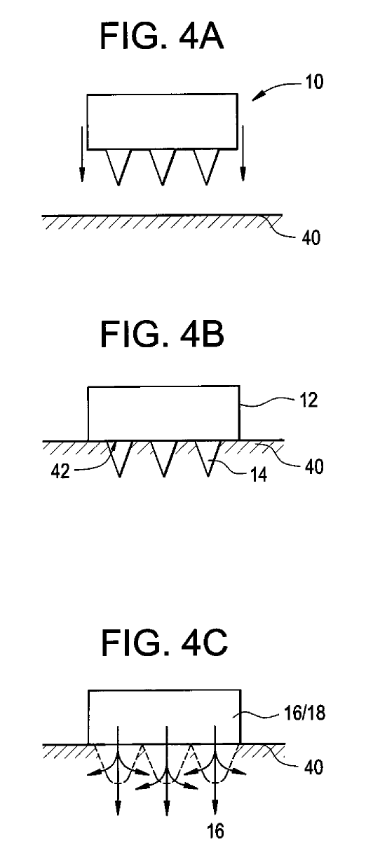

FIG. 4 illustrates one embodiment of the drug delivery method. The method generally comprises applying device 10 to biological barrier 40, to insert the array of microneedles 14 into the biological barrier 40 to create holes 42 in the biological barrier (Steps A and B). Then, the microneedles dissolve/degrade and the drug 16 diffuses from the swellable base substrate 12 through the holes 42 and into the biological barrier 40. Alternatively, the microneedles may swell without appreciable dissolution and the drug diffuses (or is driven) through the swollen microneedle.

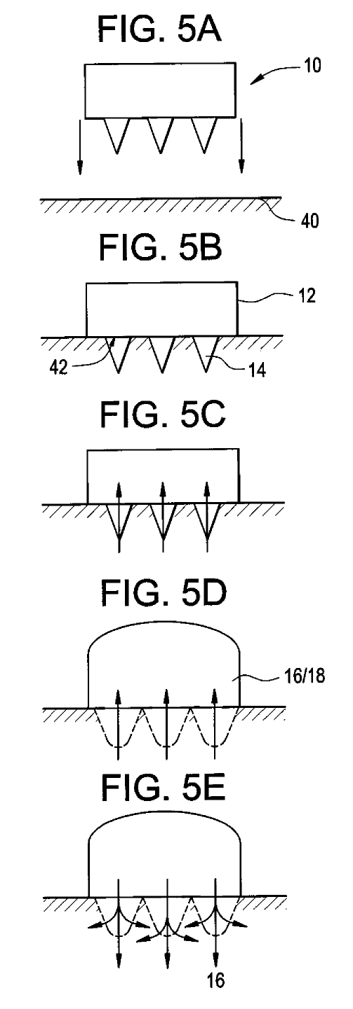

FIG. 5 illustrates another embodiment of the drug delivery method. The method generally comprises applying device 10 to biological barrier 40, to insert the array of microneedles 14 into the biological barrier 40 to create holes 42 in the biological barrier (Steps A and B). Aqueous fluids from the biological barrier flow/diffuse through the holes and/or microneedles, causing the base substrate 12 to hydrate and swell (Steps C and D). This occurs while or following dissolution of the microneedles. The drug 16 diffuses from the base substrate 12 through the holes 42 and into the biological barrier 40.

The swelling of the base substrate 12, as illustrated in FIGS. 5C and 5D, can facilitate the process of transporting materials into and out of the tissue. The swelling can also be a result of such transport processes. For example, a microneedle device can be inserted into a tissue. Interstitial fluid within the tissue can dissolve or swell the microneedles and also enter into the base substrate, which can cause the base substrate to swell. Drug present in the base substrate then transports out of the base substrate, through the pathways into the tissue created by the dissolving, or dissolved, or swollen microneedles and into the tissue.

A swellable base substrate advantageously allows a drug to be stored within the base substrate in a dry state, which typically provides a more stable environment for drug storage. When needed, the base substrate may take up fluid, such as water or interstitial fluid found in tissue, causing it to swell, which creates fluid-filled channels within the base substrate material. This swelling could, for example, create a hydrogel. In the swollen state, the base substrate can be made more permeable to the drug stored within the base substrate and thereby facilitate its transport out of the base substrate and into the tissue. Non-swellable base substrates are limited in that transport can only occur through pathways already present in the base substrate material and those pathways typically do not have a mechanism to grow in size.

The swellable base substrate material is characterized by its ability to retain its integrity when swollen. Upon swelling, the base substrate will typically changes its shape (e.g., increase its size), but preferably should not fall apart. While there may be some dissolution of base substrate material associated with the swelling, the swelling itself corresponds to a merger of the solid base substrate material and the fluid to form a solid, semi-solid or gelatinous state. It does not correspond primarily to the formation of a liquid state in which the liquid dissolves the solid base substrate material.

In contrast to the present devices and methods, a dissolvable base substrate material is characterized by its ability to enter into the fluid phase and/or be carried away with fluid contacting the base substrate material. Although, a swellable base substrate may be associated with some dissolution of base substrate material, most of the base substrate material is swollen by the fluid and is not dissolved in it.

In particular embodiments, the hydrating, degrading, or dissolving of the one or more microneedles may also provide rapid release of drug molecules dispersed or encapsulated in the microneedles. Thus, it is envisioned that embodiments of the device may provide for only the sustained release of drug molecules or for both the rapid release and sustained release of drug molecules. The sustained release may include a lag time of, for example, 1 to 12 hours, or 1 to 6 hours, or 1 to 2 hours following insertion of the microneedles into the biological tissue. A bolus release from the microneedles may be completed within one hour or another period required for complete dissolution of the microneedles.

Dissolvable microneedles advantageously create pathways for transport of material through the pathways that they create. Swellable microneedles may also be used as a means to create transport pathways, since the swellable microneedle may become more permeable upon swelling. Dissolvable microneedles are preferred, however, because they provide a more efficient transport pathway for drug release.

Dissolvable microneedles are further advantageous because after the microneedle device has been used and is removed from the tissue, there is no sharp medical waste that could pose a handling or disposal hazard. However, swellable microneedles may be preferred in other scenarios, in which there is concern about the safety of leaving dissolved microneedle material in the biological barrier.

The microneedle device is capable of delivering drug across the skin at a therapeutically useful rate. The rate of delivery may be controlled by manipulating a variety of factors, including the characteristics of the materials forming the microneedles and base substrate, the characteristics of the drug formulation to be delivered, the dimensions of each microneedle and the base substrate, and the number of microneedles in the device.

The delivery of the drug from the base substrate and microneedles into/through the barrier tissue may be enhanced by using known techniques and devices for increasing the permeability of the biological barrier and/or for augmenting molecule transport. For example, methods using electric fields (e.g., iontophoresis), ultrasound, chemical enhancers, vacuum, viruses, pH, and select application of heat and/or light may be employed in the delivery.

In another method of use, dissolvable microneedles as described herein are made wherein the microneedles and base substrate comprise no drug. After microneedle insertion and removal of the remaining substrate, a transdermal patch may be applied to the permeabilized skin.

In another aspect, the microneedle device is used for fluid extraction from the skin or other biological barrier. For example, the device may be used to collect interstitial fluid (and its solutes) from a patient, and then the fluid may be assayed for diagnostic purposes. In a particular embodiment, a method of extracting a fluid from a biological barrier is provided that includes the steps of (a) providing a microneedle device that includes (i) a base substrate which comprises a water-swellable polymeric material, and (ii) one more microneedles extending from the base substrate, which one or more microneedles comprise a water-soluble or -swellable material; (b) inserting the one or more microneedles into the biological barrier, to create a corresponding one or more holes in the biological barrier; and (c) withdrawing fluid from the biological barrier through the one or more holes and into the base substrate.

In one embodiment, the microneedle device is part of a skin patch, which can be worn by a person over a period of time, such as a few hours, a day, or a week, and then removed and the withdrawn fluid contained in the patch can be analyzed. Fluid collected is this manner could be assayed to determine its content of materials of interest, such as glucose, metabolites, drugs, toxins, and other compounds of medical, toxicological, epidemiological or other interest. This application may be particularly useful, for example, in an occupational setting to test workers for exposure to various environmental substances (e.g., potential carcinogens). In other cases, the patch can be used to test residents in a particular location for exposure to a certain biological agent of concern in that locale, for example. In other cases, the patch can be used to measure glucose concentrations to aid in therapy of diabetes. A swellable base substrate is advantageously well suited for this application, because it readily contains the fluid for real-time or subsequent analysis.

### Microneedle Fabrication Methods

In still another aspect, methods of making microneedle devices are provided. In one embodiment, the method includes a moderate-temperature, water-based fabrication process for forming the microneedles, which advantageously may be used to incorporated drug compounds that may be damaged by high processing temperatures or certain organic solvents. The methods may produce polymeric microneedle devices that have sufficient mechanical strength to penetrate the biological barrier while also being capable of rapidly degrading or dissolving within the biological barrier, for example, in less than about one hour, less than about 30 minutes, or less than 15 minutes.

In a certain embodiment, the microneedle devices described herein may be produced using a modified solvent cast-molding method. In this method, a microneedle master structure is made, for example using lithographic and etching techniques known in the art. The master structure may be an array or a single microneedle. In one case, the microneedles each have a pyramidal shape. Then, the master structure is used to make a reusable inverse mold, for example from polydimethylsiloxane. Next, a water soluble material for forming the microneedle is added into the mold in a fluidized form. For example, the water soluble material may be in an aqueous solution. Alternatively, the material may be melted, i.e., in liquid form. Alternatively, the material may be in suspension with a non-solvent liquid. A drug optionally may be included with the fluidized material. Finally, the water soluble material is hardened into the inverse shape of the microneedle mold. This hardening may include drying to remove substantially all of any solvent or non-solvent liquid used to fluidize the water soluble material. Such evaporation processes may involve increasing the temperature of the process material and/or lowering the ambient pressure, relative to room temperature and atmospheric pressure.

Alternatively, a material can be added to the mold that is chemically altered during or after molding to convert it into a water-soluble microneedle material. The chemical alteration could be conversion of monomer molecules into polymer. An example of this would be to fill the mold with liquid vinyl pyrrolidone and then polymerize the vinyl pyrrolidone by UV curing to form polyvinyl pyrrolidone as the microneedle polymeric material.

In a particular embodiment, the evaporation and/or mold filling steps may be carried out during centrifugation, vacuum or using another method capable of compacting the material to minimize or prevent the formation of voids in the microneedle. To facilitate rapid evaporation, it may be desirable to use as little solvent as feasible. While this may increase the viscosity of the material and may increase the difficulty of mold filling, centrifugation (which may involve spinning the mold) or vacuum processes may be used to forcing the fluidized material into the mold.

The base substrate may be formed simultaneously with the molding of the microneedles. In such a case, the base substrate and microneedles are integrally connected. In an alternative embodiment, all or part of the microneedles are formed in the mold, the mold surface between the microneedles is cleaned off, and then a second material is formed/molded on top of the microneedles.

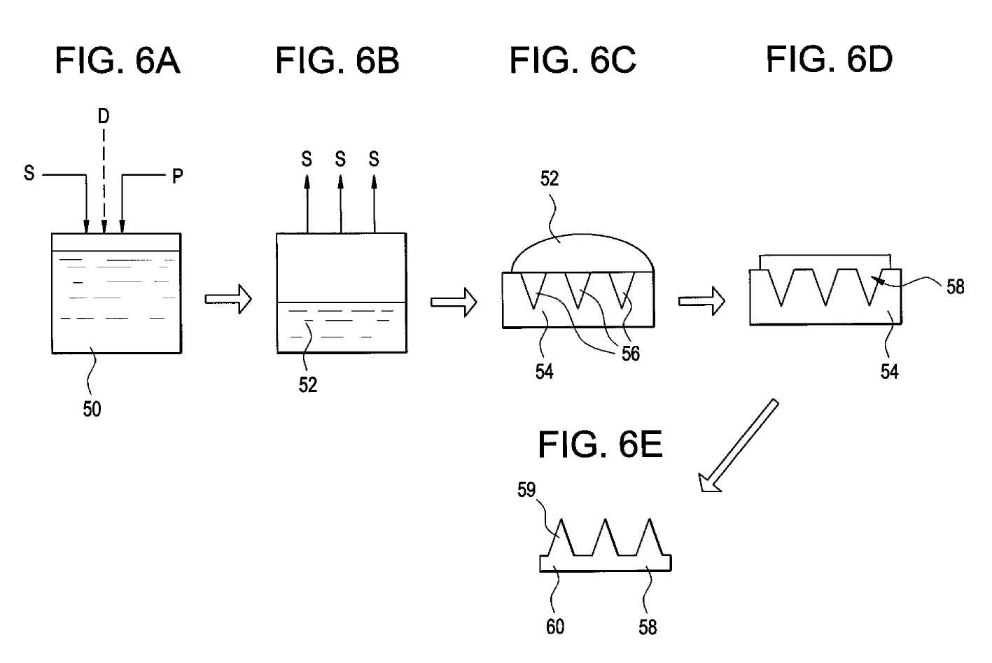

FIG. 6 illustrates one embodiment of a molding process to make a microneedle device as described herein. In Step A, a dilute solution 50 of a water-soluble material for forming the microneedle structure is made by combining the polymer or other water-soluble material (P) with an aqueous solvent (S). Optionally, a drug (D) may be added. In Step B, a concentrated solution 52 is made by evaporating a portion of the solvent. The concentrated solution may be a hydrogel. In Step C, the concentrated solution 52 is applied onto a microneedle mold 54 which includes inverse microneedle-shaped concavities 56. In Step D, centrifugal force is used to cast the device 58 in the shape of the microneedles by filling the mold cavities. In Step E, the device 58 having microneedles 59 and base substrate 60 is released from the mold. In this embodiment, a drug added to the solution 50 would result in a device 58 having drug in both the microneedle and in the base substrate.

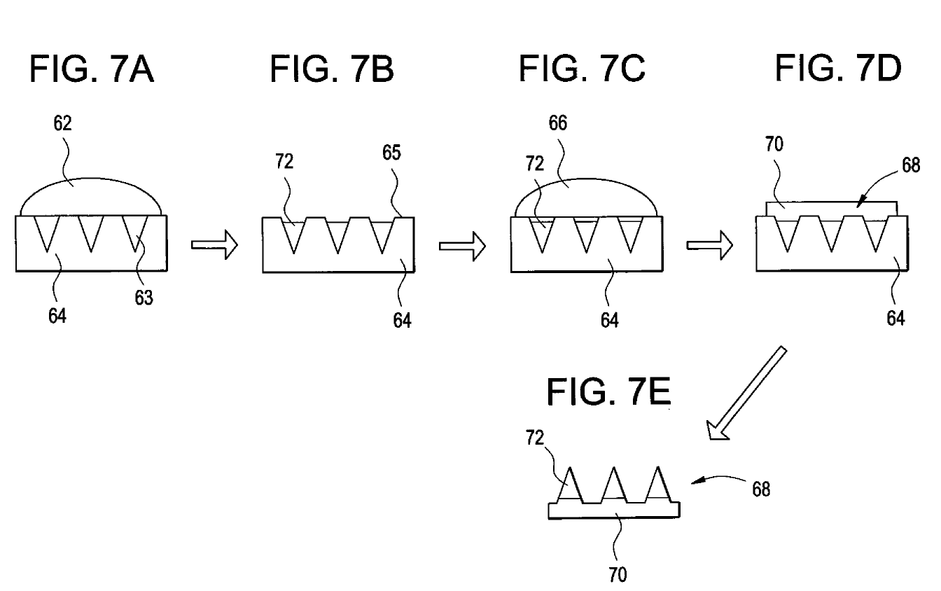

FIG. 7 illustrates another embodiment of a molding process to make a microneedle device described herein. In Step A, a first concentrated solution 62 of water-soluble material, optionally with a drug, for forming the microneedle structure (e.g., made in a like manner to that for making concentrated solution 52 as described with reference to FIG. 6) is applied onto a microneedle mold 64 which includes inverse microneedle-shaped concavities 63. In Step B, centrifugal force is used to cast the microneedles 72 by filling the mold cavities 63, and excess concentrated solution, if any, is removed from surface 65 of mold 64. In Steps C and D, a second solution 66 comprising a drug and a swellable polymeric matrix material (or precursor therefor) is applied onto the mold 64 to cast the base substrate 70 in attachment with the microneedles 72. The base substrate may be cast using centrifugal force. In Step E, the device 68 having microneedles 72 and base substrate 70 is released from the mold.

The present invention may be further understood with reference to the following non-limiting examples.

### Example 1

### Fabrication of Dissolvable Microneedles

Microneedle master structures were made using lithographic and etching techniques adapted from the microelectronics industry that are well known to those in the art. Carboxymethyl cellulose (CMC) microneedles were then fabricated using a centrifuge casting method at room temperature, as illustrated in FIG. 6.

The CMC was hydrated to form a viscous hydrogel which was placed on the surface of a mold and spun in a centrifuge at a temperature from about 25 to 40° C. The centrifugal force drove the CMC solution into the microneedle cavities in the mold. While continuing to spin the molds at elevated temperature, the water was dried from the CMC solution, leaving behind solid CMC microneedles. A model drug, sulforhodamine B fluorescent dye, was added to the viscous CMC solution and was thereby incorporated into the microneedles and into the base substrate for sustained delivery. Alternatively, the molds were filled with a solution of CMC and sulforhodamine and the mold surface wiped clean prior to placing a pure CMC solution onto the mold to form a base substrate of CMC microneedles with sulforhodamine only within the microneedles.

Compared to melting methods for polymeric microneedles, the centrifuge casting technique was able to produce perfect replicas without bubbles inside the microneedle structure. The microneedles were of a pyramidal shape having a height of about 500-600 microns and a maximum width of about 250-300 microns. The tip of the microneedle had a radius of curvature of about 25 microns.

### Example 2

### Drug Delivery with Dissolvable Microneedles

The CMC microneedles made in Example 1 were inserted by hand into full-thickness swine skin affixed to a flat surface. After fixing and sectioning, sites of microneedle insertion and drug release were imaged by brightfield and fluorescence microscopy. To quantify delivery rates, in vitro tests were performed with Franz cells containing human cadaver epidermis pierced with microneedles. Model drug release was measured by spectrofluorometry.

The CMC microneedles dissolved within 5 minutes after insertion into the swine skin. Brightfield imaging of histological sections showed the sites of microneedles insertion as an indented skin surface with a breached stratum corneum and a hole penetrating across the epidermis. Fluorescence microscopy showed intense sulforhodamine release at the sites of needle insertion. It is anticipated that if these experiments were conducted in vivo, a release in this manner near the dermal-epidermal junction would result in rapid uptake by the rich capillary bed located in the superficial dermis. Given the small size of microneedles, bolus release from an array of CMC microneedles would be expected to be particularly useful with drugs requiring sub-milligram doses.

The histological cross section of swine skin following a sustained delivery of sulforhodamine for 12 hours from the CMC microneedle device with encapsulated model drug in both the microneedles and the base substrate was evaluated (data not shown). While the microneedles rapidly hydrated and dissolved, the base substrate hydrated more slowly and caused swelling. While not wishing to be bound by any theory, it is believed that the swelling provided fluid pathways for the sulforhodamine to diffuse within the base substrate, through residual channels left by the dissolved microneedles, and into the skin.

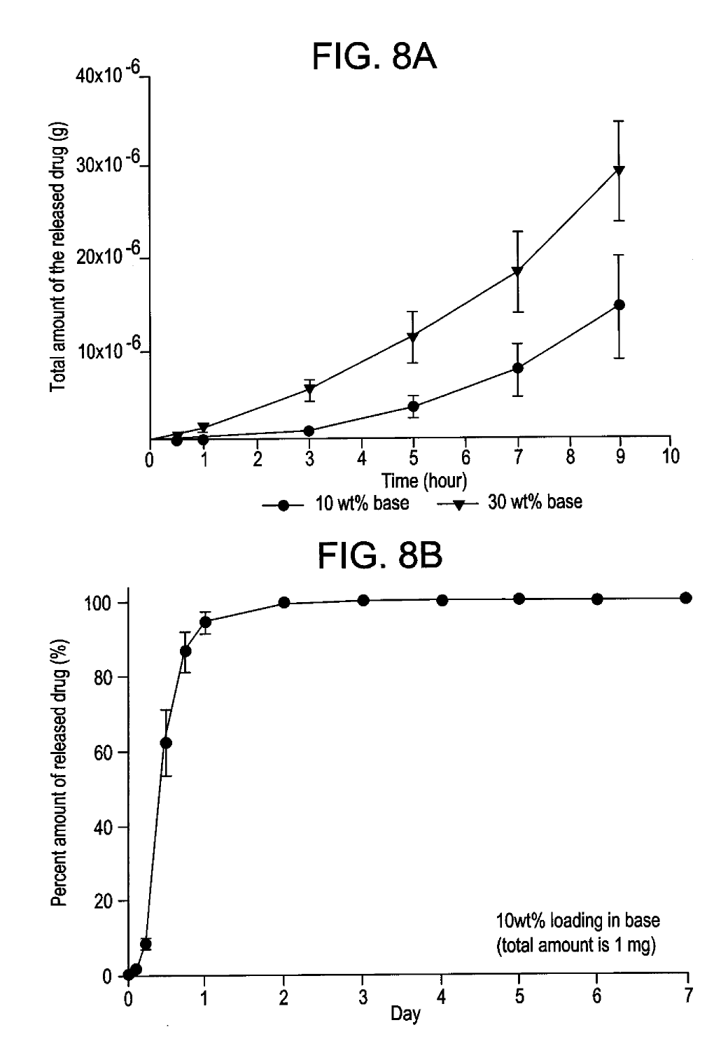

The release rates for sustained delivery are shown in FIGS. 8A-B. Although this particular example delivered drug at the microgram level, it is believed that higher loading of the base substrate of the microneedle device with drug molecules would permit delivery of milligrams of drug per day.

### Example 3

### Design and Fabrication of Microneedles by Molding

Four materials-related criteria to make microneedles for self-administration of biotherapeutics from a minimally invasive patch were considered: (1) gentle fabrication to avoid damaging sensitive biomolecules, (2) sufficient mechanical strength for insertion into skin, (3) controlled release for bolus and sustained drug delivery, and (4) rapid dissolution of microneedles made of safe materials. Guided by these criteria, two polysaccharides—i.e., carboxymethylcellulose and amylopectin—were selected because they are biocompatible materials with a history of use in FDA-approval parenteral formulations, are expected to be mechanically strong due to their relatively high Young's modulus, and are highly water soluble for rapid dissolution in the skin.

### Fabrication of Micromolds

Dissolving microneedles were fabricated using a micromolding approach that faithfully reproduces microneedle structures in an economical manner suitable for scale up to mass production. Female mastermolds were first prepared out of SU-8 photoresist by lithography and used to created PDMS male master-structures. These master-structures were then molded to make PDMS female molds. PDMS was chosen as the material for master-structures and molds because of its ability to conformally coat microstructures and fill micromolds; its poor adhesion and flexibility to facilitate separation of microstructures from micromolds; and its low cost.

Micromolds were fabricated using photolithography and molding processes. A female microneedle master-mold was structured in SU-8 photoresist (SU-8 2025, Microchem, Newton, Mass.) by UV exposure to create conical (circular cross section) or pyramidal (square cross section) microneedles tapering from a base measuring 300 μm to a tip measuring 25 μm in width over a microneedle length of 600-800 μm. A male microneedle master-structure made of polydimethylsiloxane (PDMS, Sylgard 184, Dow Corning, Midland, Mich.) was created using this mold. The male PDMS master-structure was sputter-coated (601 Sputtering System, CVC Products, Rochester, N.Y.) with 100 nm of gold to prevent adhesion with a second PDMS layer cured onto the male master-structure to create a female PDMS replicate mold. Excess PDMS on the female replicate-mold was trimmed so that the mold fit within the 27-mm inner diameter of a 50 ml conical tube (Corning Inc., Corning, N.Y.). This metal-coated male master-structure was repeatedly used to make replicate-molds that were repeatedly used to make microneedle devices.

### Fabrication of Microneedles

These micromolds were used to prepare dissolving microneedles by solvent casting with aqueous solutions of CMC and amylopectin. However, simply filling molds with CMC solution and then drying produced weak needles, probably due to structural voids left within the microneedle matrix after water evaporation. To avoid this problem, a modified casting method was developed in which the CMC solution was first concentrated by evaporation under vacuum (i.e., −50 kPa) or heating (i.e., 60-70° C.) to produce a highly viscous solution that minimized water content, but was still fluid enough to fill the mold. It was determined that an aqueous CMC solution with a viscosity of 4.5×10⁵ cP (measured with a Couette viscometer at 1/s shear rate at 23° C.) met these criteria. In case of amylopectin, the initial solvent removal was carried out at elevated temperature (i.e., 60-70° C.) rather than just under vacuum, because amylopectin has poor water solubility at room temperature.

To serve as microneedle matrix materials, ultra-low viscosity carboxymethylcellulose (CMC, Cat No. 360384, Aldrich, Milwaukee, Wis.), amylopectin (Cat No. 10120, Fluka, Steinheim, Germany) and bovine serum albumin (BSA, Sigma, St. Louis, Mo.) were dissolved in deionized water. Water was then evaporated off until the concentration of solute (e.g., CMC) was approximately 27 wt %, which resulted in a viscous hydrogel. CMC was concentrated by heating at 60-70° C. at ambient pressure or vacuuming at −50 kPa at room temperature. Amylopectin and BSA were concentrated only by the heating method at 60-70° C. or 37° C., respectively. Solute concentration was determined by measuring solution mass before and after evaporation. The viscosity of the concentrated hydrogels was measured using a Couette viscometer (Physica MCR300, Anton Paar Physica, Ostfildem, Germany). In some cases, a model drug was added by hand mixing to solubilize or suspend the compound in the concentrated hydrogel. Three model drugs were added at final concentrations of 0.15-30 wt % sulforhodamine B (Molecular Probes, Eugene, Oreg.), 20 wt % BSA (Sigma), or 5 wt % lysozyme (Sigma). The term “model drug” is used to indicate that these compounds have physicochemical and transport properties representative of certain classes of drugs, but not to suggest that these compounds have pharmacological activity representative of drugs.

To mold microneedles from concentrated hydrogels, 100-300 mg of hydrogel was placed on a female PDMS mold in a conical centrifuge tube (Corning) and centrifuged in a 45° angled rotor (GS-15R, Beckman, Fullerton, Calif.) at 3000×g and 37° C. for up to 2 h to fill the microneedle mold cavities and dry the hydrogel. The elevated temperature increased the speed of evaporation and the centrifugation continuously compressed the mold contents, which minimized void formation during drying. This modified casting method was effective to reproduce polysaccharide microneedles having the same dimensions as their master-structures for CMC and amylopectin microneedles, respectively. A similar approach was used to make microneedles out of BSA, which is a model for making needles out of pure drug, rather than encapsulating drug within a polysaccharide matrix. As an alternative approach, the same approach was attempted with high viscosity CMC (1.5-3×10³ cP for a 1% aqueous solution at 25° C.) as the matrix material, but found that it required much more water to be solubilized compared to the ultra-low viscosity CMC used above. As a result, high viscosity CMC took longer to dry and produced deformed microneedles that shrank substantially during drying and were mechanically weak.

### Fabrication of Drug-Containing Microneedles

Different drug delivery scenarios were addressed by selectively encapsulating model compounds within microneedles, within the microneedle device base substrate, or within both. To encapsulate within the CMC or amylopectin matrix, the model drug was mixed into the polysaccharide solution before casting into the molds. To selectively encapsulate within the microneedles and not in the base substrate layer, a smaller volume of drug-polysaccharide solution was cast into the holes of the micromold to form microneedles. After wiping off excess solution from the micromold surface, polysaccharide solution without model drug was cast onto the micromold and dried.

To selectively encapsulate within the base substrate and not in the microneedles, a similar two-step process was carried out, in which the model drug was only added to the polysaccharide solution applied to the micromold during the second step. Drying of the complete, integrated system or just the base substrate layer during the second step required 1-2 h, whereas drying of just the microneedles during the first step took approximately 30 min. These process times varied depending on materials and processing conditions.

To prepare microneedles with model drug encapsulated only within the microneedles and not in the base substrate layer, 8-10 mg of hydrogel mixed with model drug was filled just into the microneedle cavities in the mold and then dried under centrifugation for up to 30 min. Residual hydrogel on the surface of the mold was removed with dry tissue paper (Kimwipes, Kimberly-Clark, Roswell, Ga.) and 100-200 mg pure hydrogel (without the model drug) was then applied and dried onto the mold to form the base substrate layer. To prepare microneedles with model drug encapsulated only in the base substrate layer and not within the microneedles, the same two-step process was followed, except pure hydrogel was filled into the microneedle mold cavities and a hydrogel mixed with the model drug was used to form the base substrate layer.

### Example 4

### Microneedle Mechanical Evaluation

The design of dissolving microneedles is governed by a number of interdependent materials and fabrication constraints, one of which is the need for microneedles to have sufficient strength to insert into skin without mechanical failure. Microneedle mechanical properties were measured and simulated as a function of microneedle material composition and geometry, and then imaged insertion of optimized microneedles into skin.

### Mechanical Failure Testing

Mechanical failure tests were performed with a displacement-force test station (Model 921A, Tricor Systems Inc., Elgin, Ill., USA) on microneedles produced in accordance with Example 3. A 3×3 array containing 9 microneedles was attached to the mount of a moving sensor and an axial force was applied to move the mount at a speed of 1.1 mm/s. The mount pressed the microneedles against a flat, rigid surface of stainless steel oriented perpendicularly to the axis of mount movement. The test station recorded the force required to move the mount as a function of distance.

The mechanical behavior of CMC microneedles with a conical shape were tested first. The force-displacement curve (which is analogous to a stress-strain curve) exhibited an initial increase in force with displacement, followed by a discontinuity at a force of approximately 0.1 N/needle. This is interpreted as the point of microneedle failure, which is consistent with previous studies. Moreover, microscopic examination of the microneedles showed little deformation before this failure point and showed microneedles bent up to 90° starting approximately half way up the shaft after this failure point, which is consistent with failure by buckling. For comparison, a similar curve for PLA microneedles having the same geometry was generated, which demonstrated a five-fold greater failure force of 0.5 N/needle. Previous work showed that conical PLA microneedles similar to those used in this study have a failure force more than 3 times greater that the force needed for insertion into the skin, which indicates that these conical PLA microneedles are suitable for skin insertion without breaking. Given that the conical CMC microneedles are 5 times weaker than their PLA counterparts, this analysis suggests that the conical CMC microneedles may be too weak to insert into the skin.

Because microneedle geometry affects mechanical strength, pyramidal microneedles made of CMC and PLA were also examined. In contrast to conical microneedles, pyramidal microneedles did not show a distinct transition point indicating failure over the range of conditions tested. Microscopic examination of pyramidal microneedles showed a progressive deformation of the microneedles, starting near the tip and moving downward with increasing force, but never showed a catastrophic buckling event at a single point of failure. This progressive deformation is consistent with the continuous force-displacement curve. The reason for the different behaviors of conical and pyramidal microneedles may have to do with the larger aspect ratio and the smaller cross-sectional area of conical microneedles.

To further study the effect of microneedle composition on mechanical strength, the mechanical behavior of pyramidal microneedles having the same geometry was measured for microneedles made of CMC, PLA, amylopectin, a 20/80 wt % mixture of BSA and CMC, and 100% BSA. These five pyramidal microneedles all showed similar mechanical behavior, although the choice of material influenced microneedle strength (i.e., amount of deformation). The materials can be ranked from strongest to weakest as: PLA, amylopectin, CMC/BSA, BSA, and CMC. Amylopectin microneedles were stronger than CMC microneedles, which can be explained by the higher Young's modulus of amylopectin (4.5 GPa) compared to CMC (1 GPa). CMC and CMC/BSA microneedles were designed to simulate a CMC microneedle encapsulating a model protein and a microneedle made completely of a model protein, respectively. These two microneedles designs had similar mechanical strength, both of which were greater than for pure CMC microneedles. In this case, encapsulation of protein increased microneedle mechanical strength, but this is unlikely to be true in all cases.

### Failure Simulation Study

To better understand these experimental results, mechanical behavior of microneedles was simulated to predict critical buckling load. Critical buckling load, P_{cr}, of microneedles was simulated during axial loading using analytical methods. For the fixed-free case, where the microneedle base was fixed in position and the microneedle tip could move freely, the square-based pyramidal and circle-based conical geometries were modeled using the following equations, EQ.1 and EQ.2, respectively:

P_{cr}=E[120{H₂(H₂²(H₂−2H₁)+2H₁³)−H₁⁴}+π²{20(H₂(H₂²(−H₂+H₁)−H₁³)+H₁⁴)+π²(H₂(H₂(H₂(H₂+H₁)+H₁²)+H₁³)+H₁⁴)}]/(240π²L²) (EQ.1)

P_{cr}=E[120{R₂(R₂²(R₂−2R₁)+2R₁₃)−R₁₄}+π²{20(R₂(R₂²(−R₂+R₁)−R₁³)+R₁⁴)+π²(R₂(R₂(R₂(R₂+R₁)+R₁²)+R₁³)+R₁⁴)}]/(80πL²) (EQ.2)

Here, E is Young's modulus; L is microneedle length; H₁ and H₂ are microneedle widths at the base and tip of pyramidal microneedles, respectively; and R₁ and R₂ are radii at the base and tip of conical microneedles, respectively. Young's modulus of CMC microneedles was determined to be 1 GPa by direct measurement (MicroTester, Instron 5548, Norwood, Mass.) using bulk CMC prepared using the same casting process used to make microneedles. Young's modulus of PLA microneedles was previously determined to be 5 GPa. Tip width and diameter of pyramidal and conical microneedles, respectively, were estimated both to be 25 μm based on microscopic examination.

TABLE 1

| Conical (800 μm length) | | | Pyramidal (600 μm length) | | | Conical (600 μm length) | | |
|---|---|---|---|---|---|---|---|---|
| Diameter* | P_{cr} (N) | | Width* | P_{cr} (N) | | Diameter* | P_{cr} (N) | |
| (μm) | CMC | PLA | (μm) | CMC | PLA | (μm) | CMC | PLA |
| 50 | 0.0004 | 0.0020 | 50 | 0.0020 | 0.010 | 50 | 0.0007 | 0.0035 |
| 75 | 0.0019 | 0.0096 | 75 | 0.0087 | 0.044 | 75 | 0.0034 | 0.017 |
| 100 | 0.0061 | 0.031 | 100 | 0.026 | 0.13 | 100 | 0.011 | 0.055 |
| 200 | 0.10 | 0.51 | 200 | 0.36 | 1.8 | 200 | 0.18 | 0.91 |
| 300 | 0.5 | 2.6 | 300 | 1.8 | 8.9 | 300 | 0.94 | 4.7 |
| 400 | 1.7 | 8.4 | 400 | 5.5 | 28 | 400 | 3.0 | 15 |
| 600 | 8.6 | 43 | 500 | 13 | 67 | 500 | 7.3 | 37 |
| 800 | 27 | 137 | 600 | 27 | 137 | 600 | 15 | 76 |

As shown in Table 1, CMC microneedles with a conical geometry (800 μm length and 200 μm base diameter) have a predicted failure force of 0.10 N and PLA microneedles with the same geometry have a predicted failure force of 0.51 N, which is in excellent agreement with experimental measurements. The pyramidal microneedles (600 μm length, 300 μm base width) made of CMC and PLA have predicted failure forces of 1.8 N and 8.9 N, respectively (Table 1). The 18-fold increase in critical buckling load for these pyramidal microneedles compared to conical microneedles is also consistent with experimental measurements. However, this model accounts only for buckling and does not account for the progressive deformation observed experimentally at smaller forces.

The above comparison involved longer and thinner conical microneedles versus shorter and wider pyramidal microneedles. To make a comparison that isolates the effect just of microneedle shape, failure force for microneedles of 600 μm length and 300 μm base width/diameter was predicted to be 0.93 N and 4.7 N for conical microneedle made of CMC and PLA, respectively, which is almost two-fold smaller than the corresponding predictions for pyramidal microneedles (Table 1). It may therefore be concluded that pyramidal microneedles are stronger, probably due to their larger cross-sectional area at the same base width/diameter. Examination of Table 1 for each microneedle design as a function of base width/diameter also shows that increasing base dimensions (i.e., decreasing aspect ratio) increases needle strength. Thus, using pyramidal microneedles with a small aspect ratio can provide added mechanical strength for mechanically weak biomaterials like CMC. However, microneedles with an aspect ratio that is too small will also have poor insertion due to fabrication difficulties to make a sharp tip and insertion difficulties to force the rapidly widening needle shaft into the small hole made in the skin by the needle tip.

### Skin Insertion Testing

To determine if microneedles insert into skin, CMC pyramidal microneedles (600 μm height, 300 μm base width, and 600 μm center-to-center spacing) in a 10×10 array were inserted into full-thickness cadaver pig skin without subcutaneous fat that was shaved (series 8900, WHAL, Sterling, Ill.) and affixed under mild tension to a wooden plate using 1 cm long screws.

Microneedles were inserted by pressing against the microneedle base substrate layer with a thumb using a force of approximately 1.5 N and then removed immediately after the insertion. The site of microneedle insertion on the skin surface was exposed for 10 min to a red tissue-marking dye (Shandon, Pittsburgh, Pa., USA) that selectively stains sites of stratum corneum perforation. After wiping residual dye from the skin surface with dry tissue paper, skin was viewed by brightfield microscopy (SZX12, Olympus). Skin samples were prepared for histology by freezing in histology mounting compound (Tissue-Tek, Sakura Finetek, Torrance, Calif.) and slicing into 20-μm thick sections (Cryo-star HM 560MV, Microm, Waldorf, Germany) and then viewed by brightfield microscopy (E600, Nikon, Tokyo, Japan).

It was found that 100-needle arrays of microneedles were inserted reliably into the skin using the gentle force of a thumb. After removing the microneedles from the skin after just 3 s, the tips had already begun to dissolve indicating onset of rapid dissolution in the skin.

Histological examination of skin pierced with microneedles showed penetration depths of approximately 150-200 μm, which corresponded to insertion across the stratum corneum and viable epidermis and into the superficial dermis. Microneedles used in this experiment measured 600 μm in length, which means that one-fourth to one-third of the microneedle shaft penetrated into skin. This can be explained by deformation of skin's surface that is known to occur during microneedle insertion due to skin's viscoelasticity. The relatively wide base (i.e., 300 μm) and small aspect ratio (i.e., 2) of the pyramidal microneedles contributed to this incomplete insertion. Further optimization of microneedle geometry, such as aspect ratio, tip sharpness, and spacing between microneedles, and microneedle material may increase depth of insertion. However, as set forth in greater detail in the following examples, partial microneedle insertion is believed to be adequate for drug delivery strategies presented in this study.

### Example 5

### Drug Release from Microneedles

By loading model drug into dissolving microneedles in different ways, systems were designed that simulate bolus and extended release from a microneedle patch. To achieve bolus release, model drug was selectively incorporated into the microneedles themselves and not into the base substrate layer. In this way, the microneedles can be inserted into skin and release encapsulated drug during their rapid dissolution. The rate of release in this scenario is controlled largely by microneedle dissolution rate. A limitation is that the total dose administered is small, because microneedles each contain about 25-60 μg of matrix material and typically just a fraction of the microneedle matrix can made of drug in order to maintain microneedle mechanical strength. Thus, bolus delivery from a microneedle patch containing a few hundred microneedles is likely to be limited to less than 1 mg of drug.

To administer larger drug doses as an extended release over at least hours, model drug was incorporated into both the microneedles and base substrate layer or, alternatively, just the base substrate layer. This permits much larger doses to be administered, because the base substrate layer can be large (e.g., 10-100 mg) and can be loaded with larger fractions of drug, because base substrate layer mechanical properties have fewer constraints. In this scenario, the drug may diffuse over time from the drug reservoir in the base substrate layer and into skin through transdermal pathways created by dissolving microneedles. In this way, the base substrate layer acts as a drug source similar to a conventional matrix-design transdermal patch.

CMC pyramidal microneedles (600 μm height, 300 μm base width, and 600 μm center-to-center spacing) in a 6×6 array, produced in accordance with Example 3, were inserted by hand into pig cadaver skin. Just the microneedles, and not the base substrate layer, contained sulforhodamine B at 0.15 wt % on a dry basis, such that each microneedle contained 0.04 μg of sulforhodamine and the 36-needle array contained 1.44 μg of sulforhodamine. After 5 min, the microneedles were removed from skin and the skin sample was examined histologically. In a separate set of experiments, the shape of microneedles was also observed after 10 s, 1 min, 15 min, and 60 min insertion into the skin by light microscopy (SZX12, Olympus).

To image long-term release from dissolving microneedles into skin, sulforhodamine B was encapsulated within the needles and the base substrate layer at 0.15 wt % in a 6×6 array of CMC pyramidal microneedles (600 μm height, 300 μm base width, and 600 μm center-to-center spacing). The microneedle device contained 15 μg of sulforhodamine. The microneedles were inserted into pig cadaver skin by hand, covered with dermal tape (Blenderm, 3M Health Care, St. Paul, Minn.), and kept at room temperature for up to 12 h. Next, the microneedle device was removed and skin was examined histologically.

To quantify sulforhodamine release, a 7×7 array of CMC or amylopectin pyramidal microneedles (600 μm height, 300 μm base width, and 600 μm center-to-center spacing) was prepared with a base substrate layer of approximately 300 μm thickness. Sulforhodamine B was encapsulated within the needles and the base substrate layer at 10 wt %, which corresponded to 1 mg of sulforhodamine in the microneedle device weighing 10 mg. Alternatively, sulforhodamine was encapsulated only within the base substrate layer at 10 wt % and 30 wt %, which corresponded to almost 1 mg and 3 mg of model drug per device, respectively. Microneedles were inserted by hand into heat-stripped human cadaver epidermis (Emory University Body Donor Program, Atlanta, Ga.) with IRB approval. Microneedles were secured to skin with dermal tape and the microneedle-skin assembly was placed in a Franz diffusion chamber (Permegear, Hellertown, Pa.) at 32° C. Phosphate-buffered saline (PBS) in the receptor compartment of the Franz chamber contained 0.01 M sodium azide as an anti-bacterial agent and was sampled periodically for up to 7 days to determine sulforhodamine flux by spectrofluorimetry (QM-1, Photon Technology International, South Brunswick, N.J.).

### Bolus Release

After inserting sulforhodamine-loaded microneedles into pig cadaver skin and then removing them after 5 min, inspection of the skin surface showed an array of red spots corresponding to the sites of each microneedle insertion. These spots could not be wiped off by cleaning the skin surface and are therefore interpreted as sulforhodamine deposited within skin after microneedle dissolution.

This interpretation is confirmed by histological sections, which show deposition of sulforhodamine within skin at sites of microneedle penetration. Microneedle insertion depth was approximately 150-200 μm. The width of each hole was approximately 100 μm, which is similar to microneedle width at a distance of 150 to 200 μm up the shaft from the tip. Sulforhodamine was observed to have diffused extensively within the skin and not just at sites of microneedle insertion.

To generate a better understanding of the kinetics of bolus release from dissolving microneedles, the microneedles were imaged after insertion into skin for different times. The tips of microneedles dissolved within 10 s, half of the microneedle height disappeared within 1 min, and two-thirds disappeared within 15 min. After 1 h, microneedles were fully dissolved. The kinetics may be altered by changing microneedle geometry and matrix material. For example, it was observed that similar microneedles made of amylopectin dissolved more slowly and ones made of polyvinylpyrolide dissolved more quickly based on their different levels of water solubility. It is also worth noting that even though microneedles did not penetrate to their full length into the skin, they were nonetheless able to fully dissolve, probably due to transport of interstitial fluid from the skin up the needle shaft.

### Sustained Release

The microneedle devices designed for sustained release were inserted into skin and histological examination showed release of sulforhodamine throughout the skin.

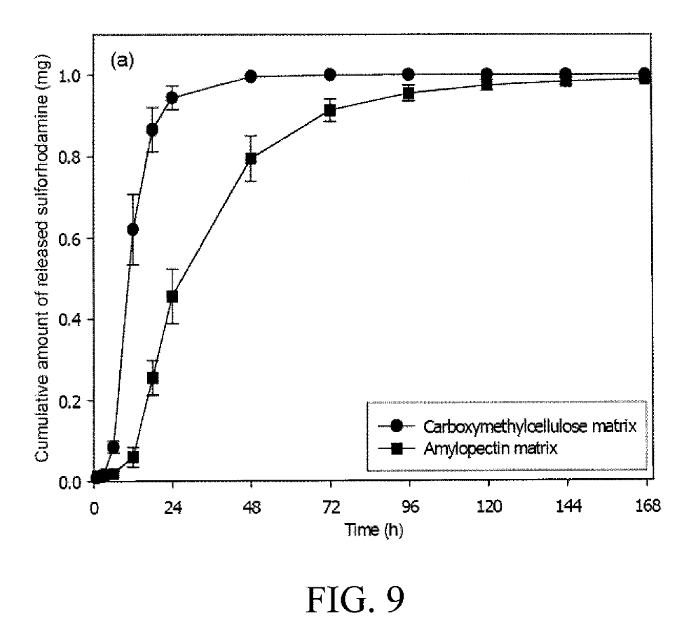

To quantify sustained release properties in greater detail, microneedle patches were inserted into human cadaver skin and transdermal flux was measured. As illustrated in FIG. 9, sulforhodamine release from CMC microneedle patches exhibited an initial lag time of a few hours, followed by steady release for approximately one day. Similar behavior was seen for microneedle patches made of amylopectin, but with slower kinetics. In this case, lag time was longer and release took place over a few days.

The data validates the hypothesis that drug encapsulated within the base substrate layer of a microneedle patch can diffuse out of the patch and into skin. Moreover, the data shows that changing microneedle patch matrix material can alter release kinetics. It is important to be able to vary release kinetics based on patch design, because different drugs administered for different indications require different release patterns.

Release rate should also depend on sulforhodamine concentration in the patch. Consistent with this expectation, the drug release rate from a patch containing 30 wt % sulforhodamine was approximately three times greater than a patch containing 10 wt % sulforhodamine.

The base substrate layer of microneedle patches was seen to swell and soften over time during sustained release delivery experiments. The dissolving microneedle patch showed extensive swelling after 15 h on the skin. As a negative control, a patch backing layer fabricated without microneedles was also placed on skin, but did not swell after placement for the same time. This suggests that the patch backing layer swelled by imbibing interstitial fluid from skin through channels created by microneedles. This observation is not only relevant to understanding drug delivery mechanisms, but also suggests uses to extract interstitial fluid for diagnostic applications, such as measuring glucose concentration in diabetics or monitoring industrial toxins in at-risk populations.

### Example 6

### Model Drug Stability and Activity

Dissolving microneedles were designed to encapsulate sensitive biomolecules using a gentle fabrication process. To assess success of this design, lysozyme was used as a model drug and changes in lysozyme's secondary structure and enzymatic activity after encapsulation and storage in CMC microneedle patches was measured.

The secondary structure of the model drug lysozyme was examined by spectropolarimetry (JASCO, J-810, Tokyo, Japan) after encapsulation and release from dissolving microneedles produced in accordance with Example 3. CMC pyramidal microneedle devices weighing 5 mg that encapsulated lysozyme at a mass fraction of 5 wt % were completely dissolved in 50 ml PBS at room temperature for 10 min and filtered by centrifugal filtration (Centricon YM-50, Millipore, Bedford, Mass., USA) at 1000×g and room temperature for 10 min to isolate lysozyme (14.3 kDa) from the dissolved CMC matrix material (90 kDa average molecular mass). After determining lysozyme concentration, PBS was added to dilute the lysozyme to 20 μg/ml. CD spectra were taken for (1) untreated lysozyme, (2) lysozyme encapsulated in microneedles that were dissolved 1 h after fabrication, (3) lysozyme encapsulated in microneedles that were dissolved after 60 days of storage at ambient conditions (23±2° C. and 38±5% relative humidity), and (4) lysozyme thermally treated at 80° C. for 30 min to cause irreversible denaturation.

Enzymatic activity of lysozyme encapsulated within CMC microneedle devices was tested with EnzCheck lysozyme assay kit (Molecular Probes). Microneedle devices weighing 5 mg that contained lysozyme encapsulated at a concentration of 5 wt % were completely dissolved in PBS at room temperature for 10 min. PBS was added to dilute each sample to 0.05 μg/ml lysozyme and 0.95 μg/ml CMC. Lysozyme activity was assayed using 1 ml solution samples for: (1) untreated lysozyme, (2) untreated lysozyme (0.05 μg) and CMC hydrogel (0.95 μg) mixed, and dissolved together, (3) lysozyme encapsulated in microneedles that were dissolved 1 h after fabrication, and (4) lysozyme encapsulated in microneedles that were dissolved after 60 days of storage at ambient conditions.

Circular dichroism (CD) analysis of untreated lysozyme compared to lysozyme encapsulated within a microneedle patch and then released by dissolution in water showed no detectable change in protein secondary structure. Even after storage of microneedle patches containing lysozyme for 2 months at room temperature, protein structure was unchanged. As a positive control, the CD spectrum showed extensive degradation of secondary structure after thermal denaturation. To further test lysozyme integrity, enzymatic activity of lysozyme was measured. To make sure that the presence of dissolved CMC after microneedle dissolution did not create an artifact, a CMC microneedle containing no lysozyme was dissolved in PBS and then mixed with untreated lysozyme. This resulted in no change in lysozyme activity.

To test the effect of encapsulation, microneedles containing encapsulated lysozyme were dissolved in PBS and found to have no loss of enzymatic activity compared to untreated enzyme. After two months of storage, lysozyme released from microneedles retained 96% enzymatic activity, indicating a small loss of activity.

### Example 7

### In Vivo Delivery of Human Growth Hormone

Microneedle devices were prepared as described in Example 3. The microneedles and base substrate were both made of CMC and, in some cases, 50% trehalose was included in the formulation too. Human growth hormone (Pfizer) was added to the microneedles at a content of 191-249 μg per 100-needle array. The ratio of growth hormone to matrix material (i.e., CMC or CMC plus trehalose) was 1 to 9. Microneedles were inserted into the skin of hairless rats (Charles River Laboratories). The microneedles inserted easily by hand and were left in place for 24 hours. Minimal skin irritation was seen after microneedle use. A positive control group was included, in which rats received subcutaneous injection of 196 μg of human growth hormone. Blood was drawn periodically and assayed using an ELISA kit specific for human growth hormone without cross-reactivity with rat growth hormone (Diagnostic Systems Laboratories, Webster, Tex.).

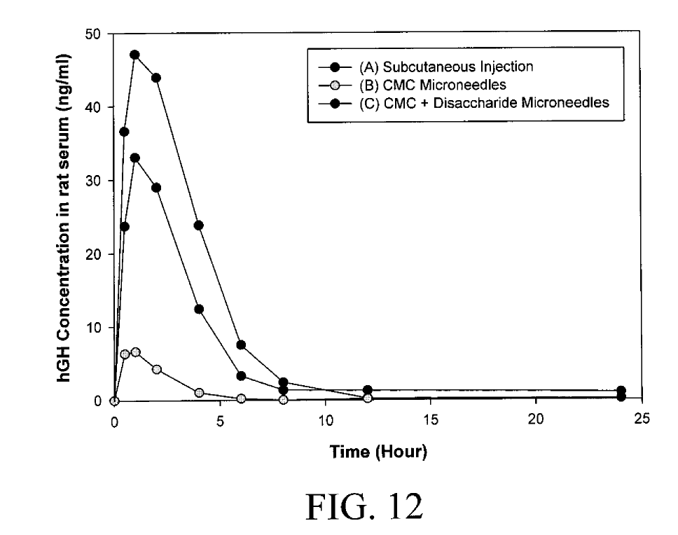

The results from this study are shown in FIG. 12. After subcutaneous injection, the serum concentration of growth hormone rapidly peaked within about one hour and then declined over the course of a few hours. Growth hormone delivery using microneedles showed a similar timecourse, although the blood levels were lower. After delivery using microneedles made of CMC with trehalose, the area under the curve was compared to the area under the curve for subcutaneous delivery and, after normalization based on the total doses applied, bioavailability was determined to be 54%, After delivery using microneedles made of CMC without trehalose, bioavailability was determined to be 9.8%. This analysis assumes that subcutaneous injection had 100% bioavailability. Overall, these data show that human growth hormone can be delivered across the skin using dissolving microneedles with a swellable base substrate and that the presence of trehalose increased bioavailability. While not wishing to be bound by any theory, it is believed that the presence of the disaccharide trehalose enabled the microneedles to be more rapidly dissolved in the skin, which facilitated release of growth hormone.

Modifications and variations of the methods and devices described herein will be obvious to those skilled in the art from the foregoing detailed description. Such modifications and variations are intended to come within the scope of the appended claims.

We claim:

1. A device for sustained delivery of drug across or into a biological barrier comprising:
a base substrate which comprises a drug dispersed in a water-swellable matrix material; and
one or more microneedles extending from the base substrate, wherein the one or more microneedles comprise a water-soluble or water-swellable material,
wherein the one or more microneedles will dissolve or swell following insertion into the biological barrier, providing a transport pathway for the drug to pass from the base substrate into the biological barrier; and
wherein the base substrate is adapted to become wetted and swell following insertion of the one or more microneedles into the biological barrier.

2. The device of claim 1, wherein the matrix material of the base substrate comprises a polymer.

3. The device of claim 1, wherein the matrix material of the base substrate comprises a water-soluble or water-swellable material.

4. The device of claim 3, wherein the water-soluble or water-swellable material of the base substrate is the same material as the water-soluble or water-swellable material of the one or more microneedles.

5. The device of claim 1, wherein the water-soluble or water-swellable material of the microneedles comprises a polysaccharide, a polysaccharide derivative, or a cellulose derivative, or a combination thereof.

6. The device of claim 1, wherein the water-soluble or water-swellable material of the microneedles becomes a hydrogel upon insertion into the biological barrier.

7. The device of claim 1, wherein the water-soluble or water-swellable material of the microneedles comprises carboxymethyl cellulose, hydroxypropylmethyl cellulose, amylopectin, starch derivatives, hyaluronic acid, or a combination thereof.

8. The device of claim 1, wherein the one or more microneedles further comprise a second drug (i) dispersed in the water-soluble or water-swellable material, (ii) coated onto the one or more microneedles, or (iii) dispersed in the water-soluble or water-swellable material and coated onto the one or more microneedles.

9. The device of claim 1, wherein the one or more microneedles are solid.

10. The device of claim 1, wherein the one or more microneedles have a length between about 10 μm and about 1500 μm and a maximum width between about 10 μm and about 500 μm.

11. The device of claim 1, wherein the one or more microneedles have a pyramidal shape.

12. The device of claim 1, further comprising a backing layer attached to the base substrate distal to the one or more microneedles.

13. The device of claim 12, wherein the backing layer having an annular region which surrounds the one or more microneedles, said region comprising an adhesive substance for contacting a patient's skin.

14. A microneedle array for drug delivery comprising,
a base substrate comprising a first drug dispersed in a swellable matrix material;
a plurality of microneedles extending from the base substrate, wherein the plurality of microneedles comprises a water-soluble or water-swellable material in which a second drug is dispersed,
wherein the plurality of microneedles will dissolve or swell following insertion into a biological barrier, providing a transport pathway for the first and second drugs to pass into the biological barrier; and
wherein the base substrate is adapted to swell following insertion of the microneedles into the biological barrier.

15. The microneedle array of claim 14, wherein the first drug and the second drug are the same drug.

16. The microneedle array of claim 14, wherein the water-soluble or water-swellable material of the plurality of microneedles comprises carboxymethyl cellulose, hydroxypropylmethyl cellulose, amylopectin, starch derivatives, hyaluronic acid, or a combination thereof.

17. The microneedle array of claim 14, wherein the matrix material of the base substrate comprises carboxymethyl cellulose, hydroxypropylmethyl cellulose, amylopectin, starch derivatives, or a combination thereof.

18. The microneedle array of claim 14, wherein the drug is a peptide, protein, or vaccine.

19. The microneedle array of claim 14, further comprises an adhesive substance coating at least a portion of the surface of the base substrate between the microneedles.

20. A method of delivering a drug across or into a biological barrier comprising:
providing a microneedle device that includes (i) a base substrate which comprises a drug dispersed in a swellable matrix material, and (ii) a plurality of microneedles extending from the base substrate;
inserting the microneedles into the biological barrier, to create a plurality of holes in the biological barrier;
wetting the base substrate and causing the base substrate to swell;
allowing the drug to diffuse from the base substrate through the holes and into the biological barrier.

21. The method of claim 20, wherein the wetting step occurs by aqueous fluids from the biological barrier flowing through the holes.

22. The method of claim 20, wherein the microneedles comprise a water-soluble or water-swellable material.

23. The method of claim 20, wherein the one or more microneedles remain substantially intact during the hydrating and swelling of the base substrate.

24. The method of claim 20, wherein the one or more microneedles further comprise a drug (i) dispersed in the water-soluble or water-swellable material, (ii) coated onto the one or more microneedles, or (iii) dispersed in the water-soluble or water-swellable material and coated onto the one or more microneedles.

25. The method of claim 24, wherein the drug from the one or more microneedles is substantially released within a period from about a few seconds to about one hour after insertion of the one or more microneedles into the biological barrier.

26. The method of claim 20, wherein the drug from the base substrate is substantially released within a period from about one hour to about seven days after insertion of the one or more microneedles into the biological barrier.

27. The method of claim 20, wherein the microneedles comprise carboxymethyl cellulose, hydroxypropylmethyl cellulose, amylopectin, starch derivatives, hyaluronic acid, or a combination thereof.

28. A method of extracting a fluid from a biological barrier comprising:
providing a microneedle device that includes (i) a base substrate which comprises a water-swellable polymeric material, and (ii) one more microneedles extending from the base substrate, which one or more microneedles comprise a water-soluble or water-swellable material;
inserting the one or more microneedles into the biological barrier, to create corresponding one or more holes in the biological barrier; and
withdrawing fluid from the biological barrier through the one or more holes and into the base substrate.

29. A method for making a microneedle device comprising:
providing an inverse mold for at least one microneedle, the mold having a base surface in which are located one or more concavities, each in the shape of a microneedle;
providing a microneedle structural material in a fluidized form, which comprises a water-soluble or -swellable material;
using centrifugation or vacuum to force the fluidized structural material into the one or more concavities;
hardening the structural material into the form of one or more microneedles;
forming a base substrate connected to the one or more microneedles, wherein the base substrate comprises a drug dispersed in a matrix material; and
releasing the one or more microneedles from the inverse mold.
